(19) United States
(12) Patent Application Publication
(10) Pub. No.: US 2015/0041734 A1
(43) Pub. Date: Feb. 12, 2015

(54) POLARIZING FILM, IMAGE DISPLAY, AND METHOD FOR PRODUCING POLARIZING FILM

(71) Applicant: NITTO DENKO CORPORATION, Ibaraki-shi, Osaka (JP)

(72) Inventors: Sadahiro Nakanishi, Ibaraki-shi (JP); Miyuki Kurogi, Ibaraki-shi (JP)

(73) Assignee: NITTO DENKO CORPORATION, Ibaraki-shi, Osaka (JP)

(21) Appl. No.: 14/370,851

(22) PCT Filed: Mar. 22, 2013
(86) PCT No.: PCT/JP2013/058311

§ 371 Date: Jul. 7, 2014

(30) Foreign Application Priority Data
Apr. 18, 2012 (JP) 2012-094444

(51) Int. Cl. G02B 5/30; G02B 1/08; C07C 309/50

(52) U.S. Cl. CPC G02B 5/3033; C07C 309/50; G02B 1/08
USPC 252/585; 534/829

(57) ABSTRACT

The present invention provides a polarizing film having a high dichroic ratio. The polarizing film includes a disazo compound represented by the following general formula (1). In the general formula (1), Q represents a substituted or unsubstituted phenyl group or a substituted or unsubstituted phenyl group having a heterocyclic ring; non-adjacent carbon atoms of the phenyl group may be substituted with nitrogen atoms; R₁ represents an alkyl group; R₂ represents a hydrogen, an acetyl group, or a substituted or unsubstituted benzoyl group; n represents 1 or 2; and M represents a counter ion.

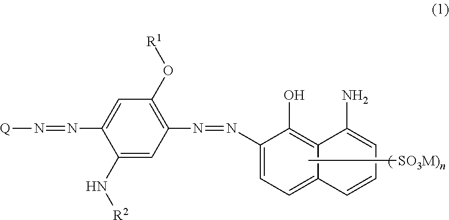

### TECHNICAL FIELD

The present invention relates to a polarizing film having a high dichroic ratio, a method for producing the polarizing film, and an image display including the polarizing film.

### BACKGROUND ART

A polarizing film is an optical member having a function to transmit specific linearly polarized light from polarized light or natural light.

General-purpose polarizing films are obtained by, for example, drawing a polyvinyl alcohol film dyed with iodine.

Polarizing films obtained by a solution casting method are also known. The solution casting method is a method of forming a polarizing film on a substrate by applying onto the substrate a coating solution containing a colorant material and a solvent.

A polarizing film obtained by a solution coating method has the advantage that its thickness is remarkably small as compared to a polarizing film obtained by drawing the polyvinyl alcohol film as described above.

Patent Document 1 discloses a polarizing film containing a disazo compound having a particular structure. However, the polarizing film in Patent Document 1 has the problem of poor transparency and a low dichroic ratio.

Furthermore, Patent Documents 2 and 3 disclose various disazo compounds used as black ink for ink-jet printers. However, these Patent Documents do not disclose about producing a polarizing film using disazo compounds.

Patent Document 1: JP 7-92531B (JP 62-330213 A)

Patent Document 2: JP 60-243157 A

Patent Document 3: JP 10-324815 A

Non-patent Document 1: Colourage, 2005, Vol. 52, Num. 2, pages 119 to 132

An object of the present invention is to provide a polarizing film having a high dichroic ratio and a method for producing the polarizing film.

The polarizing film of the present invention contains a disazo compound represented by the following general formula (1).

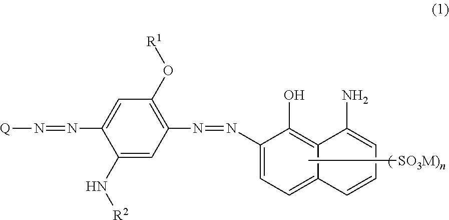

In the general formula (1), Q represents a substituted or unsubstituted phenyl group or a substituted or unsubstituted phenyl group having a heterocyclic ring, non-adjacent carbon atoms of the phenyl group may be substituted with nitrogen atoms, R¹ represents an alkyl group, R² represents a hydrogen, an acetyl group, or a substituted or unsubstituted benzoyl group, n represents 1 or 2, and M represents a counter ion.

In a preferable polarizing film of the present invention, Q is a phenyl group represented by the following general formula (2).

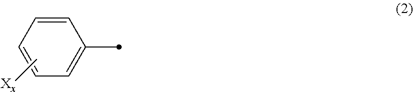

In the general formula (2), X represents an alkyl group having 1 to 6 carbon atoms, an alkoxy group having 1 to 6 carbon atoms, a thioalkyl group having 1 to 6 carbon atoms, a hydroxyalkyl group having 1 to 6 carbon atoms, an alkylamino group having 1 to 6 carbon atoms, a carboxylic acid amide group having 1 to 6 carbon atoms, a halogeno group, a nitro group, a cyano group, an amino group, an acetamide group, a carboxylic acid group, a hydroxyl group, or a —CONH₂ group; and subscript x denotes the number of substitutions of X and represents an integer of 0 to 5, and when x is 2 or more, Xs are the same or different.

In a preferable polarizing film of the present invention, X in the general formula (2) is a nitro group or a —CONH₂ group, and x in the general formula (2) is 1.

In a preferable polarizing film of the present invention, R¹ in the general formula (1) is an alkyl group having 1 to 6 carbon atoms, and R² in the general formula (1) is a hydrogen or an acetyl group.

In another aspect of the present invention, an image display is provided.

This image display includes any one of the polarizing films described above as its composition member.

In another aspect of the present invention, a method for producing a polarizing film is provided.

This method for producing a polarizing film includes a step of applying onto a substrate a coating solution containing a disazo compound represented by the above general formula (1) and a solvent.

A polarizing film of the present invention includes a disazo compound represented by the general formula (1), and therefore has a high dichroic ratio.

An image display including such a polarizing film having a high dichroic ratio is excellent in display characteristics.

### BRIEF DESCRIPTION OF THE DRAWINGS

FIG. 1 illustrates a partial sectional view showing a polarizing film according to one embodiment.

FIG. 2 illustrates a partial sectional view showing a polarizing plate according to one embodiment.

Hereinafter, the present invention is specifically described.

In the present specification, the wording “AAA to BBB” means that “AAA or more and BBB or less”. Furthermore, the wording “substituted or unsubstituted” means that “having a substituent or having no substituent”.

### [Polarizing Film]

The polarizing film of the present invention includes a disazo compound represented by the following general formula (1).

The polarizing film of the present invention includes one or more selected from disazo compounds represented by the following general formula (1), and may include other components as required.

The disazo compound represented by the general formula (1) may form a stable association in a solvent. A polarizing film formed from such a disazo compound has a high dichroic ratio.

The polarizing film of the present invention is an organic thin film formed from a coating film. The polarizing film of the present invention has a polarization property due to alignment of disazo compounds even though a special drawing treatment is not performed.

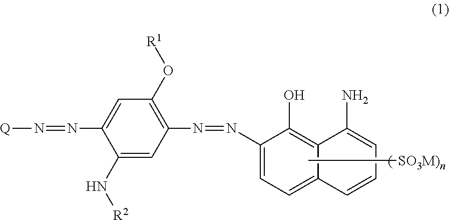

In the general formula (1), Q represents a substituted or unsubstituted phenyl group or a substituted or unsubstituted phenyl group having a heterocyclic ring; non-adjacent carbon atoms of the phenyl group may be substituted with nitrogen atoms; R¹ represents an alkyl group; R² represents a hydrogen, an acetyl group, or a substituted or unsubstituted benzoyl group; n represents 1 or 2; and M represents a counter ion.

For example, the alkyl group in R¹ is an alkyl group having 1 to 6 carbon atoms.

Hereinafter, M of —SO₃M represents a counter ion.

Examples of M (a counter ion) of —SO₃M include a hydrogen ion; an alkali metal ion such as Li, Na, K, and Cs; an alkali earth metal ion such as Ca, Sr, and Ba; other metal ions; an ammonium ion that may be substituted with an alkyl group or a hydroxyalkyl group; and a cation derived from an organic amine. Examples of an organic amine include a lower alkylamine having 1 to 6 carbon atoms, a lower alkylamine having 1 to 6 carbon atoms and a hydroxyl group, and a lower alkylamine having 1 to 6 carbon atoms and a carboxyl group.

When n in the general formula (1) is 2, each M of two —SO₃M groups may be the same or different. In addition, when M of a —SO₃M group is a divalent or higher-valent cation, M is stabilized by electrostatically bonding to other anions, or M is stabilized by being covalently held by other disazo compounds.

When the phenyl group or the phenyl group having a heterocyclic ring has a substituent, the substituent is not particularly limited, and a substituent other than a —SO₃M group is preferable. When Q has a —SO₃M group, the association of disazo compounds may be destabilized.

Examples of the substituent of the phenyl group or the phenyl group having a heterocyclic ring include an alkyl group having 1 to 6 carbon atoms, an alkoxy group having 1 to 6 carbon atoms, a thioalkyl group having 1 to 6 carbon atoms, a hydroxyalkyl group having 1 to 6 carbon atoms, an alkylamino group having 1 to 6 carbon atoms, a carboxylic acid amide group having 1 to 6 carbon atoms, a halogeno group, a nitro group, a cyano group, an amino group, an acetamide group, a carboxylic acid group, a hydroxyl group, or an aprotic polar functional group such as a —CONH₂ group. When the phenyl group or the phenyl group having a heterocyclic ring has a plurality of substituents, those substituents may be the same or different.

The number of carbon atoms for each of the alkyl group, the alkoxy group, the thioalkyl group, the hydroxyalkyl group, the alkylamino group, and the carboxylic acid amide group is preferably 1 to 4, and more preferably 1 or 2.

The halogeno group is preferably a chloro group.

The phenyl group having a substituent or the phenyl group having a heterocyclic ring and a substituent is represented by the following formula group, for example.

Q in the general formula (1) is selected from the following formula group, for example.

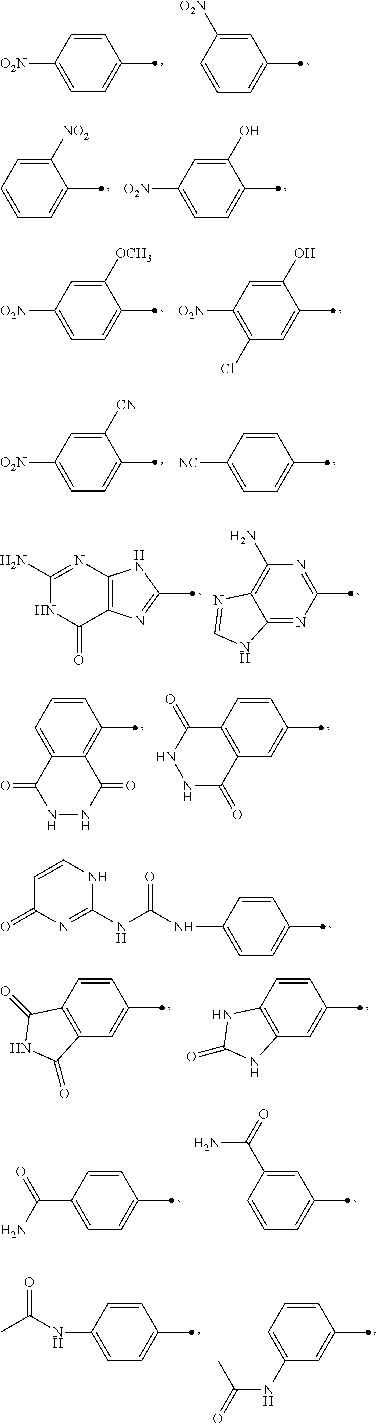

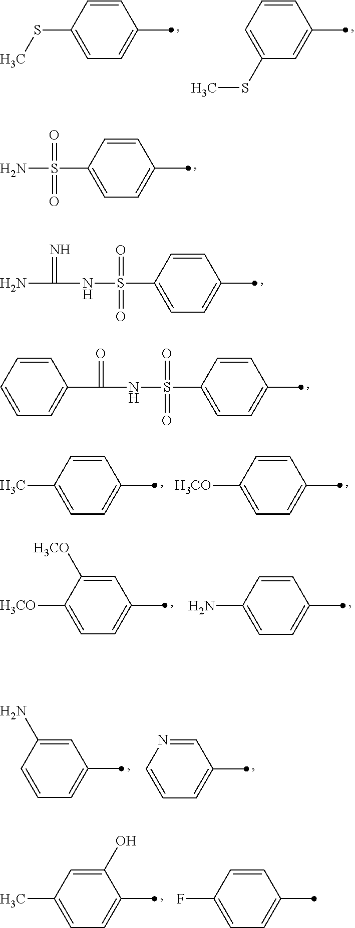

A disazo compound having Q which is a substituted or unsubstituted phenyl group is preferably used since a more stable association can be formed.

The substituted or unsubstituted phenyl group in Q is represented by the following general formula (2).

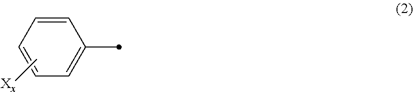

In the general formula (2), X represents an alkyl group having 1 to 6 carbon atoms, an alkoxy group having 1 to 6 carbon atoms, a thioalkyl group having 1 to 6 carbon atoms, a hydroxyalkyl group having 1 to 6 carbon atoms, an alkylamino group having 1 to 6 carbon atoms, a carboxylic acid amide group having 1 to 6 carbon atoms, a halogeno group, a nitro group, a cyano group, an amino group, an acetamide group, a carboxylic acid group, a hydroxyl group, or a —CONH₂ group, and subscript x denotes the number of substitutions of X and represents an integer of 0 to 5. When x is 2 or more, Xs are the same or different.

The number of carbon atoms for each of the alkyl group, the alkoxy group, the thioalkyl group, the hydroxyalkyl group, the alkylamino group, and the carboxylic acid amide group is preferably 1 to 4, and more preferably 1 or 2.

The halogeno group is preferably a chloro group.

X in the general formula (2) is preferably a nitro group (—NO₂) or —CONH₂ group because a polarizing film having a higher dichroic ratio can be obtained. x in the general formula (2) is preferably 1 (the substituent X is one).

In particular, a disazo compound in which Q is a phenyl group selected from the following formula group is preferably used.

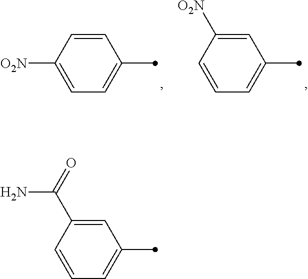

R¹ of the general formula (1) is preferably an alkyl group having 1 to 4 carbon atoms, and more preferably an alkyl group having 1 or 2 carbon atoms.

The alkyl group exemplified as the substituent of the phenyl group, the alkyl included in such as the alkoxy group exemplified as the substituent of the phenyl group, and the alkyl group represented by R¹ each may be either linear or branched.

When the benzoyl group represented by R² in the general formula (1) has a substituent, examples of the substituent include the substituent of the phenyl group.

R² in the general formula (1) is preferably a hydrogen or an acetyl group.

n of (SO₃M)ₙ in the following general formula (1) is preferably 2.

In the general formula (1), the bonding position of the SO₃M with the naphthyl group is not limited.

In the general formula (1), the naphthyl group to which the SO₃M attached is selected from the following formula group, for example.

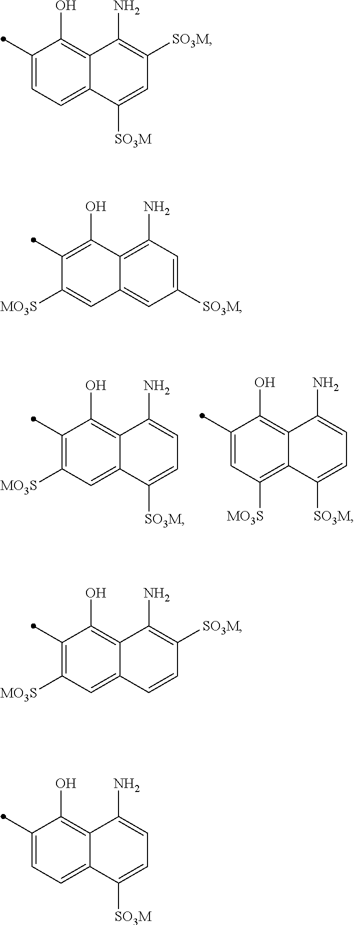

The disazo compounds represented by the general formula (1) can be synthesized in accordance with “Riron Seizoh, Senryo Kagaku (Theory Production, Dye Chemistry), 5^{th} Edition” written by Yutaka Hosoda (published by Gihodo Shuppan Co., Ltd. on Jul. 15, 1968, pages 135 to 152).

For example, a monoazo aniline compound is obtained by converting a substituted or unsubstituted aniline compound into a diazonium salt, and subjecting the salt to a coupling reaction with an aminonaphthalenesulfonic acid compound. After the monoazo aniline compound is converted into a diazonium salt, the above-mentioned disazo compound represented by the general formula (1) can be obtained by carrying out a coupling reaction with an aminohydroxylnaphthalene disulfonic acid under weakly alkaline.

After obtaining a disazo compound in which R² is an acetyl group according to the above method, a disazo compound in which R² of the general formula (1) is a hydrogen can be obtained by hydrolyzing the obtained compound with an acid or an alkali.

The polarizing film of the present invention includes a disazo compound represented by the above general formula (1), and the content thereof is not particularly limited. For example, the content of the disazo compound in the polarizing film is 50% by mass to 100% by mass, and preferably 80% by mass to 100% by mass.

The polarizing film of the present invention may include other components in addition to the disazo compound represented by the above general formula (1). Examples of the other component include a disazo compound other than those of the general formula (1), a monoazo compound, a colorant other than an azo compound, a polymer, and an additive. Examples of the additive include a compatibilizer, a surfactant, a heat stabilizer, a light stabilizer, a lubricant, an antioxidant, a flame retardant, and an antistatic agent.

The content of the other components is not particularly limited, and is, for example, more than 0% by mass and not more than 50% by mass, and preferably more than 0% by mass and not more than 20% by mass.

The polarizing film of the present invention has an absorption dichroism at least at partial wavelength between the wavelength of 380 nm and 780 nm. The dichroic ratio of the polarizing film is preferably 15 or more, and more preferably 20 or more. The dichroic ratio is obtained by methods described in the following Examples. According to the present invention, a polarizing film having a dichroic ratio of 20 or more can be provided.

The degree of polarization of the polarizing film of the present invention is preferably 97% or more, more preferably 98% or more, and particularly preferably 99% or more. The degree of the polarization can be adjusted according to a thickness of the film, for example.

The transmittance of the polarizing film (measured at a wavelength of 550 nm and 23° C.) is preferably 35% or more, and more preferably 40% or more.

The degree of polarization and the transmittance can be measured using a spectrophotometer (product name: “V-7100”, manufactured by JASCO Corp.).

The thickness of the polarizing film of the present invention is not particularly limited, and it is preferably 0.05 μm to 5 μm and more preferably 0.1 μm to 1 μm.

As to the reason why a polarizing film including a disazo compound represented by the general formula (1) has a high dichroic ratio, the present inventors assume the following theories.

When the disazo compound of the general formula (1) is dissolved or dispersed in a solvent, a plurality of disazo compounds form an association in the liquid. When a plurality of disazo compounds form an association, substituents of disazo compounds having a molecular structure of the general formula (1) bond at a position where those substituents are hard to sterically repel with each other. Accordingly, the plurality of disazo compounds associate well planarly, and a polarizing film formed from the stable association has a high dichroic ratio.

### [Coating Solution and Method for Producing Polarizing Film According to the Present Invention]

The method for producing a polarizing film according to the present invention includes a step of applying onto a substrate a coating solution containing a disazo compound represented by the above general formula (1) and a solvent. Here, “applying” means that a coating film is formed by flow-casting or applying a liquid onto a substrate.

The method for producing a polarizing film according to the present invention includes the above-described step as a prerequisite, and may include other steps. For example, the production method may include a step of drying a coating film obtained from the above-described step. The substrate is preferably a substrate given an alignment control force.

The polarizing film of the present invention can be produced through, preferably the following steps A and B; and after the step B, the following step C may be performed:

step A: a step of forming a coating film by applying a coating solution containing the disazo compound and a solvent on a substrate.

step B: a step of drying the coating film.

step C: a step of performing water-resistant treatment to the surface of the coating film which is dried in the step B.

The coating solution includes a disazo compound represented by the general formula (1) and a solvent that can dissolve or disperse the disazo compound. The disazo compounds used are not particularly limited as long as they are included in the general formula (1), and the compounds may be selected to use alone or in combination of two or more kinds.

The coating solution is obtained by dissolving or dispersing the disazo compounds in a solvent such as an aqueous solvent.

Other components other than the disazo compound may be added to the solvent as necessary.

The solvent is not particularly limited, and the conventionally known solvent may be used, but an aqueous solvent is preferable. Examples of the aqueous solvent include water, a hydrophilic solvent, and a mixed solvent containing water and the hydrophilic solvent. The hydrophilic solvent is a solvent, which can be dissolved with water substantially uniformly. Examples of the hydrophilic solvent include alcohols such as methyl alcohol, ethyl alcohol, and isopropyl alcohol; glycols such as ethylene glycol, and diethylene glycol; cellosolves such as methyl cellosolve, and ethyl cellosolve; esters such as acetic ether; and the like. Preferably, water or the mixed solvent containing water and the hydrophilic solvent is used for the aqueous solvent.

The coating solution exhibits a liquid crystal phase by changing liquid temperature or concentration of the disazo compounds. That is, the disazo compound represented in the general formula (1) is a lyotropic liquid crystalline compound.

This liquid crystal phase is exhibited by the disazo compounds forming an association in a solvent. The liquid crystal phase is not particularly limited, and examples of the liquid crystal phase include a nematic liquid crystal phase, a smectic liquid crystal phase, a cholesteric liquid crystal phase, and a hexagonal liquid crystal phase. The liquid crystal phase can be identified and confirmed from an optical pattern when observed by using a polarization microscope.

The concentration of the disazo compounds in the coating solution is not particularly limited, but the concentration of which the disazo compounds are not precipitated is preferable. The concentration of the solution may be set at which the liquid crystal phase is exhibited or not exhibited. The concentration of the disazo compounds in the coating solution is preferably 0.05% by mass to 50% by mass, more preferably 0.5% by mass to 40% by mass, and particularly preferably 2% by mass to 30% by mass.

The pH of the coating solution is appropriately prepared. The pH of the coating solution is preferably pH 2 to 10, and more preferably pH 6 to 8.

Further, the coating solution is prepared at the temperature of preferably 10° C. to 40° C., and more preferably 15° C. to 30° C.

The coating film may be formed by applying the coating solution on the surface of an appropriate substrate.

The substrate is used for uniformly developing the coating solution. The type of the substrate is not particularly limited as far as it is proper to the object. Examples of the substrate include a polymer film, a glass plate, and a metal plate. Hydrophilic treatment such as corona treatment may be performed for the substrate surface on which the coating solution is to be coated.

An aligned film may be used as the polymer film. Since the aligned film has an alignment control force on its surface, the disazo compounds in the liquid can be surely aligned. An aligned film can be obtained by giving an alignment control force to the film, for example. Examples of the method for giving an alignment control force include: subjecting the surface of the film to a rubbing treatment; forming a film of polyimide or the like on the surface of the film, and subjecting the surface of the film to a rubbing treatment; forming a film composed of a photoreactive compound on the surface of the film, and irradiating the film with light to form an alignment layer; and the like.

Preferably, a polymer film such as an aligned film is used as a substrate, and a polymer film excellent in transparency (for example, having a haze value of 3% or less) is preferably used.

As a material for the polymer film, polyester type such as polyethylene terephthalate; cellulose type such as triacetylcellulose; polycarbonate type; acryl type such as polymethyl methacrylate; styrene type such as polystyrene; olefin type such as polypropylene, and polyolefin having a ring-shaped or a norbornen structure; and the like can be cited. A norbornen type film is preferably used to align the disazo compounds sufficiently.

The method for applying a coating solution is not particularly limited, and for example, an application method using a previously known coater may be employed. Examples of the coater include a bar coater, a roll coater, a spin coater, a comma coater, a gravure coater, an air knife coater, and a die coater.

When the coating solution is applied to the surface of a substrate, shearing stress is applied to an association of disazo compounds in the process of flowing of the coating solution. Accordingly, the longitudinal direction of the association becomes parallel to the flow direction of the coating solution, so that a coating film in which the association of the disazo compounds is aligned can be formed on the substrate. In the coating solution of the present invention, the disazo compounds form a stable association, and therefore a coating film in which the disazo compounds substantially uniformly are aligned can be formed.

For enhancing alignment of the disazo compounds, a magnetic field, an electric field, or the like may be applied as required after the coating film is formed.

After the coating solution is applied to form a coating film, the uncured coating film is dried.

Drying of the uncured coating film can be performed by natural drying, forced drying, or the like. Examples of the forced drying include drying under reduced pressure, drying by heating, drying by heating under reduced pressure, and the like.

The concentration of the disazo compounds increases in the process of drying in the drying step, so that aligned disazo compounds are fixed. When alignment of the disazo compounds in the coating film is fixed, absorption dichroism, a property of the polarizing film, is generated. The obtained dry coating film can be used as a polarizing film.

The polarizing film of the present invention can be formed by a solution casting method using the coating solution as described above. Thus, an extremely thin polarizing film can be produced according to the present invention. The thickness of the obtained polarizing film is, for example, 0.05 μm to 10 μm, preferably 0.05 μm to 5 μm, and more preferably 0.1 μm to 1 μm.

For imparting water resistance to the surface of the dried coating film, the following treatment may be performed.

Specifically, a solution containing at least one compound salt selected from the group consisting of an aluminum salt, a barium salt, a lead salt, a chromium salt, a strontium salt, a cerium salt, a lanthanum salt, a samarium salt, an yttrium salt, a copper salt, an iron salt, and a compound salt having two or more amino groups per molecule is brought into contact with the surface of the dry coating film.

By performing this treatment, a layer containing the compound salt is formed on the surface of the dry coating film. By forming such a layer, the surface of the dry coating film can be made insoluble or hardly soluble in water. Accordingly, water resistance can be imparted to the dry coating film (polarizing film).

The surface of the obtained polarizing film may be washed with water or a cleaning liquid as required.

### (Use of Polarizing Film of the Invention)

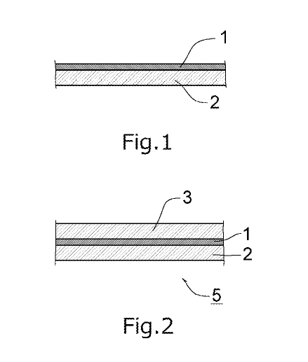

A polarizing film 1 obtained by applying the coating solution on the substrate is laminated on a substrate 2 as shown in FIG. 1.

The polarizing film 1 of the present invention is generally used in the state that the polarizing film 1 is laminated on the substrate 2 such as a polymer film. However, the polarizing film 1 can be used by peeling off from the substrate 2.

Other optical film may be further laminated on the polarizing film 1 of the present invention. Examples of the other optical film include a protective film, and a retardation film. A polarizing plate may be composed by laminating the protective film and/or the retardation film on the polarizing film of the present invention.

A polarizing plate 5 on which a protective film 3 is laminated to the polarizing film 1 of the present invention is illustrated in FIG. 2. This polarizing plate 5 includes the substrate 2 such as a polymer film, the polarizing film 1 laminated on the substrate 2, and the protective film 3 laminated on the polarizing film 1. The substrate 2 has a function to protect the polarizing film 1. Thus, the polarizing plate 5 includes the protective film 3 laminated on only one surface of the polarizing film 1.

Further, although it is not particularly illustrated in Figures, other optical film such as a retardation film may be laminated on the polarizing plate 5.

When any other optical film is laminated to the polarizing film, any appropriate adhesive layer is provided therebetween from a practical point of view. Examples of the material that forms the adhesive layer include an adhesive, a pressure sensitive adhesive, an anchor coating agent, and the like.

Use of the polarizing film of the present invention is not particularly limited. The polarizing film of the present invention can be used for, for example, a composition member of image displays such as a liquid crystal display, an organic EL display.

When the image display is a liquid crystal display, the preferable use thereof is a TV set, a portable device, a video camera, and the like.

### EXAMPLES

The present invention is further described using Examples as follows. However, the present invention is not limited to only these Examples below. Further, methods for each analysis used in Examples are as follows.

### [Method for Measuring Dichroic Ratio of Polarizing Film]

A spectrophotometer (product name: “V-7100”, manufactured by JASCO Corp.) equipped with a Glan-Thompson polarizer was used, and k1 and k2 of Y values subjected to vision sensitivity correction is calculated by radiating a measuring beam of a linearly polarized light into a polarizing film which is an object to be measured. The values k1 and k2 are substituted for the following equation, thereby calculating the dichroic ratio. Here, k1 represents the transmittance of the linearly polarized light in the maximum transmittance direction of the polarizing film, and k2 represents the transmittance of the linearly polarized light in the direction perpendicular to the maximum transmittance direction.

Dichroic ratio=log(1/k2)/log(1/k1) Equation:

### [Observation Method of Liquid Crystal Phase]

A small amount of a coating solution obtained by a dropper made from polyethylene was sandwiched between two glass slides and a liquid crystal phase was observed by using a polarization microscope (product name: “OPTIPHOT-POL,” manufactured by Olympus Corporation) equipped with a large-scale sample heating/cooling stage for microscopes (product name: “10013L”, manufactured by Japan High Tech Co. Ltd.).

### [Measurement of Thickness of Polarizing Film]

A portion of the polarizing film formed on a norbornen type polymer film was peeled off from the polymer film and a step between the polymer film and the polarizing film was measured by using a three-dimensional non-contact surface form measuring system (product name: “Micromap MM5200,” manufactured by Ryoka Systems Inc.).

### Example 1

4-nitroaniline (1 equivalent) was formed into a diazonium salt using sodium nitrite (1 equivalent) and hydrochloric acid (5 equivalents), and the diazonium salt was subjected to a coupling reaction with 3′-amino-4′-methoxyacetanilide (1 equivalent) in a weakly-acidic cold aqueous solution, thereby obtaining a monoazo compound. The monoazo compound (1 equivalent) was formed into a diazonium salt using sodium nitrite (1 equivalent) and hydrochloric acid (2.5 equivalents), and the diazonium salt was subjected to a coupling reaction with sodium 4-amino-5-hydroxy-1,3-naphthalenedisulfonic acid (another name: SS acid) (0.95 equivalents) in a weakly-basic cold aqueous solution, thereby obtaining a disazo compound. For converting the sulfonic acid salt of the disazo compound into a lithium salt, the disazo compound was salted out with lithium chloride to thereby obtain the disazo compound represented by the following formula (A).

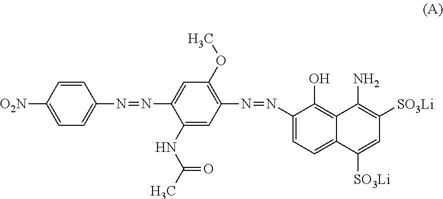

A coating solution having a disazo compound concentration of 25% by mass was prepared by dissolving the disazo compound of the above formula (A) in ion-exchanged water. This coating solution having a concentration of 25% by mass was observed at 23° C. in accordance with the method for observing a liquid crystal phase as described above, and was found to show a nematic liquid crystal phase.

The coating solution was diluted by further adding ion-exchanged water, thereby preparing a coating solution having a disazo compound concentration of 5% by mass.

A bar coater (product name: “Mayer rot HS4”, manufactured by Bushman Co.) was used to apply the coating solution having a concentration of 5% by mass onto the surface of a norbornene type polymer film (product name: “ZEONOR”, manufactured by Nippon Zeon Co., Ltd.) subjected to rubbing treatment and corona treatment, and the resultant coating film was naturally dried. The dried coating film is the polarizing film.

The thickness of the obtained polarizing film was about 0.2 μm.

The dichroic ratio of the polarizing film was measured in accordance with the method for measuring a dichroic ratio as described above. The measurement result is shown in Table 1.

### Example 21

The disazo compound represented by the following formula (B) was obtained in the same manner as in Example 1 except that 3-nitroaniline was used instead of 4-nitroaniline, and sodium 4-amino-5-hydroxy-2,7-naphthalenedisulfonic acid (another name: H acid) was used instead of sodium 4-amino-5-hydroxy-1,3-naphthalenedisulfonic acid.

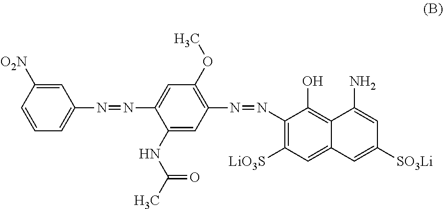

A coating solution having a disazo compound concentration of 25% by mass was prepared by dissolving the disazo compound of the above formula (B) in ion-exchanged water. This coating solution having a concentration of 25% by mass was observed at 23° C. in accordance with the method for observing a liquid crystal phase as described above, and was found to show a nematic liquid crystal phase.

The coating solution was diluted by further adding ion-exchanged water, thereby preparing a coating solution having a disazo compound concentration of 5% by mass.

A polarizing film was prepared in the same manner as in Example 1 using this coating solution. The thickness of the obtained polarizing film was about 0.2 μm.

The measurement result of a dichroic ratio of the polarizing film is shown in Table 1.

### Example 31

A disazo compound represented by the following formula (C) was obtained in the same manner as in Example 1 except that 3-aminobenzamide was used instead of 4-nitroaniline, and sodium 4-amino-5-hydroxy-2,7-naphthalenedisulfonic acid (another name: H acid) was used instead of sodium 4-amino-5-hydroxy-1,3-naphthalenedisulfonic acid.

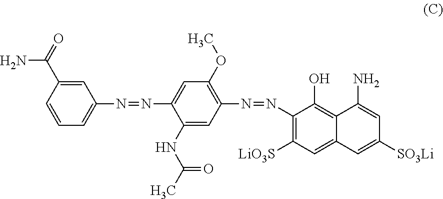

A coating solution having a disazo compound concentration of 25% by mass was prepared by dissolving the disazo compound of the above formula (C) in ion-exchanged water. This coating solution having a concentration of 25% by mass was observed at 23° C. in accordance with the method for observing a liquid crystal phase as described above, and was found to show a nematic liquid crystal phase.

The coating solution was diluted by further adding ion-exchanged water, thereby preparing a coating solution having a disazo compound concentration of 5% by mass.

A polarizing film was prepared in the same manner as in Example 1 using this coating solution. The thickness of the obtained polarizing film was about 0.2 μm.

The measurement result of a dichroic ratio of the polarizing film is shown in Table 1.

### Example 4

A salt of sulfonic acid of a disazo compound was obtained by hydrolyzing the disazo compound of the formula (C) obtained in Example 3 for one hour at 80° C. using an excessive amount of sodium hydroxide. For converting the sulfonic acid salt into a lithium salt, the disazo compound was salted out with lithium chloride to thereby obtain the disazo compound represented by the following formula (D).

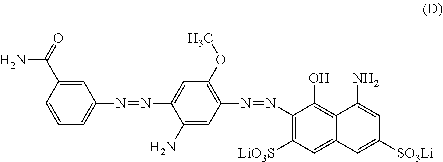

A coating solution having a disazo compound concentration of 25% by mass was prepared by dissolving the disazo compound of the above formula (D) in ion-exchanged water. This coating solution having a concentration of 25% by mass was observed at 23° C. in accordance with the method for observing a liquid crystal phase as described above, and was found to show a nematic liquid crystal phase.

The coating solution was diluted by further adding ion-exchanged water, thereby preparing a coating solution having a disazo compound concentration of 5% by mass.

A polarizing film was prepared in the same manner as in Example 1 using this coating solution. The thickness of the obtained polarizing film was about 0.2 μm.

The measurement result of a dichroic ratio of the polarizing film is shown in Table 1.

TABLE 1

| | Disazo compound | Dichroic ratio |
|---|---|---|
| Example 1 | Formula (A) | 25.2 |
| Example 2 | Formula (B) | 36.0 |
| Example 3 | Formula (C) | 35.8 |
| Example 4 | Formula (D) | 33.5 |

### [Evaluation]

Polarizing films in Examples 1 to 4 all had high dichroic ratios.

In particular, polarizing films in Examples 2 to 4 had higher dichroic ratios.

### INDUSTRIAL APPLICABILITY

The polarizing film of the present invention can be used for, for example, a composition member of a liquid crystal display, a polarizing sunglass, and the like.

The coating solution of the present invention can be used as a forming material for a polarizing film.

1. Polarizing film, 2. Substrate, 3. Protective film, 5. Polarizing plate

1. A polarizing film comprising a disazo compound represented by the following general formula (1): 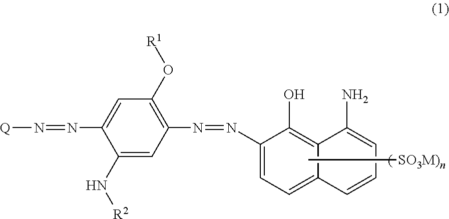
wherein Q represents a substituted or unsubstituted phenyl group or a substituted or unsubstituted phenyl group having a heterocyclic ring; non-adjacent carbon atoms of the phenyl group may be substituted with nitrogen atoms; R¹ represents an alkyl group; R² represents a hydrogen, an acetyl group, or a substituted or unsubstituted benzoyl group; n represents 1 or 2; and M represents a counter ion.

2. The polarizing film according to claim 1, wherein Q is a phenyl group represented by the following general formula (2): 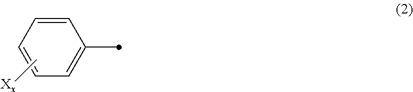
wherein X represents an alkyl group having 1 to 6 carbon atoms, an alkoxy group having 1 to 6 carbon atoms, a thioalkyl group having 1 to 6 carbon atoms, a hydroxyalkyl group having 1 to 6 carbon atoms, an alkylamino group having 1 to 6 carbon atoms, a carboxylic acid amide group having 1 to 6 carbon atoms, a halogeno group, a nitro group, a cyano group, an amino group, an acetamide group, a carboxylic acid group, a hydroxyl group, or a —CONH₂ group; and subscript x denotes the number of substitutions of X and represents an integer of 0 to 5, and when x is 2 or more, Xs are the same or different.

3. The polarizing film according to claim 2, wherein X in the general formula (2) is a nitro group or a —CONH₂ group, and x in the general formula (2) is 1.

4. The polarizing film according to claim 1, wherein R¹ in the general formula (1) is an alkyl group having 1 to 6 carbon atoms, and R² in the general formula (1) is a hydrogen or an acetyl group.

5. An image display comprising the polarizing film according to claim 1.

6. A method for producing a polarizing film comprising a step of applying onto a substrate a coating solution containing a disazo compound represented by the following general formula (1) and a solvent, 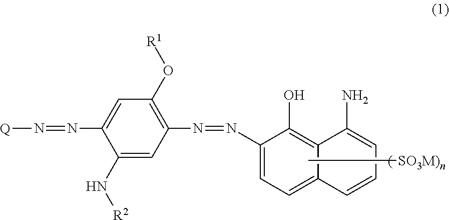
wherein Q represents a substituted or unsubstituted phenyl group or a substituted or unsubstituted phenyl group having a heterocyclic ring; non-adjacent carbon atoms of the phenyl group may be substituted with nitrogen atoms; R¹ represents an alkyl group; R² represents a hydrogen, an acetyl group, or a substituted or unsubstituted benzoyl group; n represents 1 or 2; and M represents a counter ion.
